(19) United States
(12) Patent Application Publication
(10) Pub. No.: US 2026/0337649 A1
(43) Pub. Date: Oct. 29, 2026

(54) Process for the Production of a Purified Human Milk Oligosaccharide Derived from a Microbial Fermentation Process

(71) Applicant: Chr. Hansen A/S, Hoersholm (DK)

(72) Inventor: Tobias SEITZ, Rheinbreitbach (DE)

(73) Assignee: Chr. Hansen A/S, Hoersholm (DK)

(21) Appl. No.: 19/103,897

(22) PCT Filed: Aug. 28, 2023
(86) PCT No.: PCT/EP2023/073563

§ 371 Date: Feb. 14, 2025

(30) Foreign Application Priority Data
Aug. 29, 2022 (EP) 22192607.4

(51) Int. Cl. C12P 19/04

(52) U.S. Cl. CPC C12P 19/04

(57) ABSTRACT

Disclosed is a process for the production of a human milk oligosaccharide (HMO) derived by a microbial fermentation method, the process comprising the steps of: a) providing a fermentation broth comprising at least one HMO and biomass; b) adding a multivalent cation salt to the fermentation broth; and c) subjecting the mixture of step b) to centrifugation.

The present invention relates to a process for the production of a purified human milk oligosaccharide by microbial fermentation. More specifically, the present invention relates to facilitating the separation of residual biomass and other solid contaminants from the fermentation broth by centrifugation.

### BACKGROUND

Human breast milk contains substantial amounts of carbohydrates. The carbohydrates that are present in human breast milk include monosaccharides such as L-fucose and N-acetylneuraminic acid, the disaccharide lactose, and up to 20 g/l oligosaccharides, the so-called “human milk oligosaccharides (HMOs)”. HMOs represent the third most abundant constituent of human breast milk. It is presumed that more than 150 structurally distinct oligosaccharides are present in human milk. Selected HMOs are shown in Table 1. Each of about 10 to 13 of these HMOs are present in human milk in a concentration of between several hundred milligrams to grams per liter (Thurl et al., (2017), Nutrition Reviews 75(11) 920-933). Among the HMOs, neutral HMOs are known as well as acidic HMOs which contain at least one N-acetylneuraminic acid (NeuAc) moiety. The structural complexity and abundance of these oligosaccharides is unique for human milk and has not been found in the milk of other mammals such as—for example—domesticated dairy animals.

Since HMOs are not digested by humans, the physiological role of these saccha-rides has been under investigation for several decades. The prebiotic effect of HMOs was discovered more than 100 years ago. Upon consumption, HMOs are able to modulate the composition of the human gut microbiome by supporting growth of beneficial bacteria.

TABLE 1
Structures of notable oligosaccharides in human breast milk.

| Name | Chemical structure |
|---|---|
| 2′-FL | Fuc(α1, 2)Gal(β1, 4)Glc |
| 3-FL | Gal(β1, 4)(Fuc(α1, 3))Glc |
| DFL | Fuc(α1, 2)Gal(β1, 4)Glc |
| | (α1, 2)\| |
| | Fuc |
| LNT | Gal(β1, 3)GlcNAc(β1, 3)Gal(β1, 4)Glc |
| LNnT | Gal(β1, 4)GlcNAc(β1, 3)Gal(β1, 4)Glc |
| LNFP-I | Fuc(α1, 2)Gal(β1, 3)GlcNAc(β1, 3)Gal(β1, 4)Glc |
| LNFP-II | Gal(β1, 3)(Fuc(α1, 4))GlcNAc(β1, 3)Gal(β1, 4)Glc |
| LNFP-III | Gal(β1, 4)(Fuc(α1, 3))GlcNAc(β1, 3)Gal(β1, 4)Glc |
| LNFP-V | Gal(β1, 3)GlcNAc(β1, 3)Gal(β1, 4)(Fuc(α1, 3))Glc |
| 3′-SL | NeuAc(α2, 3)Gal(β1, 4)Glc |
| 6′-SL | NeuAc(α2, 6)Gal(β1, 4)Glc |
| LSTa | NeuAc(α2, 3)Gal(β1, 3)GlcNAc(β1, 3)Gal(β1, 4)Glc |
| LSTb | NeuAc |
| | \|(α2, 6) |
| | Gal(β1, 3)GlcNAc(β1, 3)Gal(β1, 4)Glc |
| LSTc | NeuAc(α2, 6)Gal(β1, 4)GlcNAc(β1, 3)Gal(β1, 4)Glc |
| LNDFH-I | Fuc(α1, 2)Gal(β1, 3)GlcNAc(β1, 3)Gal(β1, 4)Glc |
| | \|(α1, 4) |
| | Fuc |
| LNDFH-II | Gal(β1, 3)GlcNAc(β1, 3)Gal(β1, 4)Glc |
| | \|(α1, 4)(α1, 3)\| |
| | Fuc Fuc |
| LNDFH-III | Gal(β1, 3)GlcNAc(β1, 3)Gal(β1, 4)Glc |
| | \|(α1, 3)(α1, 3)\| |
| | Fuc Fuc |
| F-LSTa | NeuAc(α2, 3)Gal(β1, 3)GlcNAc(β1, 3)Gal(β1, 4)Glc |
| | \|(α1, 4) |
| | Fuc |
| F-LSTb | NeuAc |
| | \|(α2, 6) |
| | Fuc(α1, 2)Gal(β1, 3)GlcNAc(β1, 3)Gal(β1, 4)Glc |
| F-LSTc | NeuAc(α2, 6)Gal(β1, 4)GlcNAc(β1, 3)Gal(β1, 4)Glc |
| | (α1, 3)\| |
| | Fuc |
| FSL | NeuAc(α2, 3)Gal(β1, 4)Glc |
| | (α1, 3)\| |
| | Fuc |
| DS-LNT | NeuAc |
| | \|(α2, 6) |
| | NeuAc(α2, 3)Gal(β1, 3)GlcNAc(β1, 3)Gal(β1, 4)Glc |
| FDS-LNT-I | NeuAc |
| | \|(α2, 6) |
| | NeuAc(α2, 3)Gal(β1, 4)GlcNAc(β1, 3)Gal(β1, 4)Glc |
| FDS-LNT-II | NeuAc |
| | \|(α2, 6) |
| | NeuAc(α2, 3)Gal(β1, 4)GlcNAc(β1, 3)Gal(β1, 4)Glc |
| | (α1, 3)\| |
| | Fuc |

Several other functional effects of HMOs were revealed in the last years, especially their effect on the development of neonates. HMOs are known to act as decoys to reduce the risk of infections by bacterial and viral pathogens which adhere to human cells by binding to these cells' surface glycoproteins. Additionally, various HMOs possess an anti-inflammatory effect and act as immunomodulators. Hence, it was proposed that HMOs reduce the risks of developing food allergies. A positive effect of sialylated HMOs on the development of a neonate's central nervous system is also intensely discussed (reviewed in “Prebiotics and Probiotics in Human Milk, Origins and functions of milk-borne oligosaccharides and bacteria”, Academic Press (2017) editors: McGuire M., McGuire M., and Bode L.).

To take advantage of the beneficial effects of HMOs, efforts are being made in adding individual HMOs to nutritional composition, in particular to infant formula.

The limited supply of HMOs for supplementing nutritional compositions led to the development of procedures for chemical syntheses of HMOs. The drawbacks in such chemical syntheses lead to biocatalytic approaches wherein HMOs are synthesized in vitro using purified enzymes such as glycosyltransferases. Today, individual HMOs are produced in an industrial scale using fermentation of gene-tically-engineered microbial cells (WO 2015/150328 A1, WO 2017/043382 A1, WO 2010/070104 A1, WO 2012/097950 A1). The HMOs are synthesized by genetically-engineered microbial cells and can be recovered from the fermentation medium and/or cell lysate to obtain a substantially pure preparation of the HMO.

During their recovery from a fermentation broth, HMOs are usually present in form of a liquid process stream, e.g. an aqueous solution which contains the HMO of interest and which in a vital first step of the purification process has to be separated from the solid residual phase of the fermentation broth. This separation generally is accomplished either by filtration methods or by centrifugation in a first step.

In large scale production, filtration methods alone tend to prove complex because of the necessity of possible clogging of the filtration membranes and the need for repetitious filtration steps to make sure that a majority of the solid phase is being captured by the filter medium without impairing the yield of the HMO. At present, a relatively good fermentation clarified liquid can be obtained by using a method of combining a deep filtration system with more than 5 layers of deep filtration membranes for processing cell fermentation liquid, but the method has limited treatment capacity and higher cost, and greatly limits the large-scale application of the deep filtration system in production.

On the other hand, centrifugation methods are widely known and applied in large industrial scale production. For instance, the disc centrifuge is widely applied to the fields of fruit juice beverages, tea beverages, wine brewing, seasonings, edible oil, plant protein, animal protein, fish processing, biopharmaceuticals, starch processing, industrial fermentation, yeast extraction, natural extraction and the like, is widely applied to large-scale industrial production, and the working mode of continuous treatment is very suitable for large-scale cell fermentation production in the field of biopharmaceuticals. However, when a centrifugal clarification treatment on the fermentation broth by a disc centrifuge at the fermentation end point is carried out as a first separation step, the clarified supernatant still contains a plurality of components, such as a few cells, colloidal substances, cell debris and the like. Because the efficiency of the centrifuge separation step is vital for all the subsequent purification steps necessary to achieve a food-grade product, especially in large scale production there is still the need for improvement of the centrifugation of fermentation broth in the production of HMOs.

Aiming at the problems of low treatment capacity, high turbidity, large dosage of a deep filtration membrane and higher treatment cost in the prior fermentation liquor pretreatment clarification process, the present invention aims to provide a facilitated method for treating HMO producing microbial fermentation broth by a centrifuge system, so as to remove more efficiently various impurities in the fermentation liquor, such as cell debris, host DNA, proteins and the like, obtain the clarified fermentation liquor containing target HMO with less remaining solid parts, thus further simplify the process steps required by downstream HMO purification and reduce the process production cost.

The object is achieved by a process for the production of a human milk oligosaccharide (HMO) derived by a microbial fermentation method, the process comprising the steps of:
a) providing a fermentation broth comprising at least one HMO and biomass;
b) adding a multivalent cation salt to the fermentation broth; and
c) subjecting the mixture of step b) to centrifugation.

### SUMMARY

In a first aspect, a process for the production of a purified human milk oligosaccharide (HMO) derived by a microbial fermentation method is provided.

In a second aspect, the use of a multivalent cation salt as additive to a fermentation broth at the end of a microbial fermentation process in the production of a human milk oligosaccharide is provided.

### DETAILED DESCRIPTION

According to the first aspect, a process is provided for the production of a human milk oligosaccharide (HMO) derived by a microbial fermentation method, the process comprising the steps of:
a) providing a fermentation broth comprising at least one HMO and biomass;
b) adding a multivalent cation salt to the fermentation broth; and
c) subjecting the mixture of step b) to centrifugation.

Presently and as generally understood in the related field, a “microbial fermentation method” is to be understood as a—generally large—scale-industrial metabolic process where during cultivation of microorganisms such as bacteria, fungi and moulds, enzymatic decomposition and utilization of nutrients, particularly carbohydrates, and the conversion of compounds into other compounds takes place. As such, industrial or large-scale microbial fermentation is the process of controlling microorganisms, i.e. bacteria, yeast, and moulds, to modify food, producing a desired product.

Presently, and generally in the relevant field, a “microorganism” presently designates and encompasses any microscopic organism that comprises either a single cell, cell clusters, or multicellular relatively complex organisms, which is suitable to be employed in the process according to the invention, and particularly includes bacteria and yeast. A microorganism as employed according to the invention can be cultivated in a liquid medium, and generally needs a carbon source in the medium to grow and replicate.

Presently, and throughout the invention, “recombinant” means genetically engineered DNA prepared by transplanting or splicing genes from one species into the cells of a host microorganism of a different species. Such DNA becomes part of the host's genetic makeup and is replicated.

Consequently, “a host microorganism” is designated to mean any microorganism containing nucleic acid sequences or expressed proteins foreign to/not naturally occurring in the thus recombinant host microorganism and wherein the foreign/not naturally in said microorganism occurring nucleic acid sequence is integrated in the genome of the host microorganism cell. Thereby, “not naturally occurring” means that the nucleic acid sequence/protein (such as, e.g. an enzyme), is foreign to said host microorganism cell, i.e. the nucleic acid sequences/proteins are heterologous with respect to the microorganism host cell. The heterologous sequence may be stably introduced, e.g. by transfection, transformation, or transduction, into the genome of the host microorganism cell, wherein techniques may be applied which will depend on the host cell the sequence is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). Thus, the host microorganism the heterologous sequence has been introduced in will produce the heterologous proteins the nucleic acid sequences according to the invention are coding for.

For recombinant production, host cells can be genetically engineered to incorporate expression systems or portions thereof and the nucleotide sequences encoding enzymes necessary for the metabolic pathways and/or biosynthesis of the human milk oligosaccharide of interest. Introduction of a nucleic acid sequence into the host microorganism cell can be effected by methods described in many standard laboratory manuals, such as Davis et al., Basic Methods in Molecular Biology, (1986), and Sambrook et al., 1989, supra.

A great variety of expression systems can be used to produce the HMOs of the invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and to synthesize a HMO in a host may be used for expression in this regard. The appropriate DNA sequence may be inserted into the expression system by any of a variety of well-known and routine techniques, such as, for example, those set forth in Sambrook et al., supra.

Presently, a “desired oligosaccharide” designates an oligosaccharide that shall specifically and intentionally be produced with the applied process. Accordingly, a “non-desired oligosaccharide” represents an oligosaccharide not intended to be produced or generated during the production of the desired oligosaccharide or an oligosaccharide formed by the used organism unrelated to the desired oligosaccharide. A “metabolic product” or “metabolic saccharide product” or “saccharide intermediate” is a saccharide, i.e. carbohydrate product generated during production of a desired oligosaccharide, and an “unused saccharide substrate” represents a starting molecule or moiety used for/during the production of the desired oligosaccharide.

As mentioned at the outset, the invention relates to a process for producing a desired oligosaccharide using a host microorganism, wherein said desired oligosaccharide is not naturally occurring in said host cell and is a human milk oligosaccharide. Reference in this connection is made the HMOs listed in table 1 above.

With the process according to the invention, an effective manufacturing method has been provided, by means of which a desired oligosaccharide can be produced in a form where non-desired solids or otherwise hindering solid products or unused solid substrates from the fermentation broth are essentially no longer present.

As used herein, the term “cultivating” means growing a microorganism in a medium and under conditions permissive and suitable for the production of the desired human milk oligosaccharide. A couple of suitable host microorganisms as well as the mediums and conditions for their cultivation will be readily available for one skilled in the art upon reading the disclosure of this invention in connection with the skilled person's technical and expert background.

In step a) of the disclosed inventive process a fermentation broth is provided comprising at least one HMO and biomass from the fermentation process.

The desired HMO, such as 2′-fucosyllactose (2′-FL), 3-fucosyllactose (3-FL), 3′-sialyllactose (3′-SL), Lacto-N-tetraose (LNT), or 6′-sialyllactose (6′-SL), is produced by fermentation of a genetically modified microbial organism. Fermentation may be performed in any suitable fermentation medium, such as, for example, a chemically defined fermentation medium.

The fermentation medium may vary based on the microbial organism used and the desired HMO. The fermentation medium can be supplemented with trace minerals and vitamins such as calcium pantothenate, biotin, nicotinic acid, myo-inositol, thiamine HCl, p-aminobenzoic acid. Food grade processing aids such as antifoam and pH control agents can also be used. The temperature of the fermentation step is typically 10 to 50 °C, preferably 25 to 35 °C, more preferably 28 to 32 °C. The pH of the fermentation step is typically 3 to 8, preferably, 5 to 7, more preferably 5.5 to 6.5. At the conclusion of the fermentation step, the fermentation broth can be subjected to a heat treatment step to deactivate the microbe prior to the downstream treatment steps.

Preferably, the microbial organism is a genetically modified yeast such as a Saccharomyces strain, a Candida strain, a Hansenula strain, a Kluyveromyces strain, a Pichia strain, a Schizosaccharomyces stain, a Schwanniomyces strain, a Torulaspora strain, a Yarrowia strain, or a Zygosaccharomyces strain. More preferably, the yeast is Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pom be, Schwanniomyces occidentalis, Torulaspora delbrueckii, Yarrowia lipolytica, Zygosaccharomyces rouxii, or Zygosaccharomyces bailii.

Preferably, the microbial organism can also be selected from the genera E. coli or S. cerevisiae, Bifidobacterium, Lactobacillus, Enterococcus, Streptococcus, Staphylococcus, Peptostreptococcus, Leuconostoc, Clostridium, Eubacterium, Veilonella, Fusobacterium, Bacterioides, Prevotella, Escherichia, Propionibacterium and Saccharomyces, Bifidobacterium adolescentis, B. animalis, B. bifidum, B. breve, B. infantis, B. lactis, B. longum; Enterococcus faecium; Escherichia coli, Klyveromyces marxianus; Lactobacillus acidophilus, L. bulgaricus, L. casei, L. crispatus, L. fermentum, L. gasseri, L. helveticus, L. johnsonii, L. paracasei, L. plantarum, L. reuteri, L. rhamnosus, L. salivarius, L. sakel, Lactococcus lactis (including but not limited to the subspecies lactis, cremoris and di acetyl actis); Leuconostoc mesenteroides (including but not limited to subspecies mesenteroides); Pedicoccus acidilactici, P. pentosaceus; Propionibacterium acidipropionici, P. freudenreichii ssp. shermanii; Staphylococcus carnosus; and Streptococcus thermophilus.

A desired HMO, such as 2′-fucosyllactose (2′-FL), 3-fucosyllactose (3-FL), 3′-sialyllactose (3′-SL), Lacto-N-tetraose (LNT), or 6′-sialyllactose (6′-SL), is purified from a fermentation broth that is a product of the fermentation process. After fermentation, the broth containing the desired HMO is applied to the separation process as summarized below.

The term “fermentation broth”, as used in this specification, refers to the product obtained from fermentation of the microbial organism. Thus, the fermentation product comprises biomass (cells), the fermentation medium, residual substrate material, and any molecules/by-products produced during fermentation, such as the desired HMO. After each step of the purification method, one or more of the components of the fermentation product is removed, resulting in a more purified HMO.

Presently, and throughout the invention, “biomass” means each and any kind of cell or microorganism substance, cell or microorganism debris, high molecular weight molecules, and/or residual lysis products as well as other solid particles adhering thereto or being present in the fermentation broth to be treated in the subsequent separation steps of the present inventive process.

Optionally, the fermentation broth can be subjected to a heat treatment step to kill bacteria or other undesired micro-organisms that may be present to any significant extent.

Typically, the desired HMO is present in the liquid or in other words the supernatant phase of the fermentation broth. However, it is also encompassed in the sense of the present invention that the desired HMO is present or contained in the biomass fraction of the fermentation broth.

In step b) of the process of the present invention a multivalent cation salt is added to the fermentation broth.

As used herein, the term “multivalent cation” refers to a cation capable of forming multiple ion pairs, e.g., a multiply charged cation, such as a double charged or a triply charged cation. Any convenient multivalent cations may find use in the subject salt compositions. In some embodiments, the multivalent cation is divalent. Divalent cations of interest include, but are not limited to, magnesium, zinc and calcium. In some embodiments, the multivalent cation is trivalent. Trivalent cations of interest include, but are not limited to, aluminium. In certain embodiments of the salt composition, the at least one multivalent cation is selected from the group consisting of magnesium, zinc, aluminium and calcium. In certain embodiments of the salt composition, the at least one multivalent cation is magnesium. In certain embodiments of the salt composition, the at least one multivalent cation is zinc. In certain embodiments of the composition, the at least one multivalent cation is aluminium. In certain embodiments of the composition, the at least one multivalent cation is calcium.

In the sense of the present invention the term “multivalent cation salt” is directed to any multivalent cation composition including a suitable counteranion. The counteranion can be single valent, such as e.g. a chloride, bromide, fluoride, acetate, or it can be multivalent, e.g. divalent or trivalent such as for example sulphate, sulphite, phosphate, phosphonate, carbonate.

The selection of the multivalent cation salt can vary depending on the solubility, stability and possible interaction with the other compositions present in the fermentation broth.

The multivalent cation salt can be added to the fermentation broth at any time before the centrifugation step c) for separating the biomass from the supernatant phase is started. Preferably, the multivalent cation salt is added to the fermentation broth shortly before the subsequent centrifugation step is started.

The multivalent cation salt can be added in any form suitable especially for large-scale industrial production, e.g. as solid or in the form of an aqueous solution or dispersion.

In step c) of the method of the present invention the mixture of step b), i.e. the fermentation broth comprising the desired HMO and biomass with the added multivalent cation salt is subjected to a centrifugation. Like this, some, most or all of the solid fractions of the fermentation broth are separated from the liquid supernatant phase.

Centrifugation technologies mainly exploit differences in size, density, specific gravity, and shape and are well known and established in the present field of biomanufacturing. Typically, filters are simpler to operate as they do not have any moving parts while centrifuges have rotating machinery. On the other hand, filters offer slower processing, are prone to clogging, have higher operating costs, and generally require large surface areas and floor space as compared to centrifuges.

Most centrifugation technologies for biomanufacturing are based on tubular bowl or disc-stack. Tubular bowl technology is mostly used for microbial cultures as it can generate high centrifugal force (>10,000 g) and can produce paste of solids with low supernatant content. Once the solids fill the bowl, the spinning bowl needs to be stopped to discharge solids. Disc-stack (Westfalia CSV series) and countercurrent (e.g. kSep, Elutra, and Rotea) centrifuges do not pack the cells tightly but form a slurry and intermittently discharge the solids without requiring them to stop during discharges. Countercurrent centrifugation technologies exert low shear, but other bowl or disc-stack centrifugation technologies exert high shear forces and therefore are not used when intact cells are required as a product or an intermediate product. Thus, the type of centrifugation technology can be readily chosen by a skilled person in dependence of the desired production parameters.

It was surprisingly found that the addition of a multivalent cation salt to the fermentation broth before the broth is subjected to a centrifugation separation step leads to a more effective separation. Notably, there can be seen an increase in the amount of solid phase accumulating during centrifugation as well as a lowering in turbidity of the liquid supernatant phase. Like that, the multivalent cation salt acts as a separation aiding additive, also making the subsequent purification steps to result in the pure HMO product easier and faster.

Without being bound to any theory, it is assumed that the multivalent cation salt aids in the precipitation of remaining biomass and other solid remains of the fermentation process, possibly by increasing the size of the precipitate or disturbing the tendency of any residual solid from the fermentation process of finely dispersing in the fermentation medium, thereby overall increasing the efficiency of the centrifugation.

In other words, according to the present invention a process is provided for the production of a human milk oligosaccharide (HMO) derived by a microbial fermentation method, the process comprising the steps of:
a) providing a fermentation broth comprising at least one HMO and biomass from the fermentation; and
b) subjecting the mixture of step a) to centrifugation,
   wherein a multivalent cation salt is added to the fermentation broth of step a) before the centrifugation of step b) is started.

In another embodiment the present disclosure provides for a process as described above wherein the HMO is selected from the group consisting of 2′-FL, 3-FL, LNT, LNnT, LNFPI, LNFPII, LNFPIII, LNFPV, 3′-SL, 6′-SL, LST-a, LST-b, LST-c and DSLNT.

For the named HMOs good results in the improved separation quality of the centrifugation separation step can be achieved. Reference is made to the structures of the HMOs as shown in Table 1 presented above.

In another embodiment the present disclosure provides for a process as described above wherein said multivalent cation salt is a divalent or trivalent cation salt, preferably selected from the group consisting of Mg salts Ca salts, Sr salts, Zn salts, Al salts, Fe salts, Mn salts, and Cu salts and mixtures thereof.

As counteranions of the said divalent or trivalent cation salts monovalent, divalent, trivalent or tetravalent anions can be present, preferably monovalent or divalent anions.

Typically, such salts are chosen that are well known in the field of biomolecular production and thus not only readily available but also certified for the use in biomolecular production of food-grade products, like e.g. chlorides, phosphates, phosphonates, sulfites, or sulphates of Mg, Ca, Fe and Zn.

Another vital aspect in the choice of the multivalent cation salt is the ease to remove the salt from the HMO containing phase, either the solid biomass phase or preferably the liquid supernatant phase separated by the centrifugation step. In the subsequent further purification steps the nature of the multivalent cation salt plays a role in choosing a well-known and large-scale enabling separation method that in the best case would be carried out in any event, even without the presence of the multivalent cation salt. Preferably, the multivalent cation salt can be removed from the HMO containing phase in a subsequent work-up step by ion exchange using ion exchange absorption resins, ion exchange bed filtration, ion exchange column filtration, or related techniques (chromatography).

In another embodiment the present disclosure provides for a process as described above wherein the multivalent cation salt is selected from the group consisting of MgCl₂, ZnCl₂, and CaCl₂).

These salts have a good solubilty in water and thus can be easily handled in metering the amount added in form of an aqueous solution to the fermentation broth. Moreover, the cations and anions of the salts can be easily removed in a subsequent work-up step by ion exchange methods. In addition, the salts are readily available in a high grade quality and typically are certified to be safe for the use in biomolecular production of food-grade substances.

In another embodiment the present disclosure provides for a process as described above wherein the multivalent cation salt is added to the fermentation broth of step a) in form of an aqueous solution.

Like that, easy handling in metering the amount added to the fermentation broth can be achieved as well as easing a continuous large scale operation of the inventive method.

In another embodiment the present disclosure provides for a process as described above wherein the concentration of the multivalent cation salt in the fermentation broth after addition of the multivalent cations to the fermentation broth (step b)) is ≥0.05 weight-%, preferably ≥0.1 weight-%, more preferably ≥0.15 weight-%, and most preferably ≥0.175 weight-%; and ≤0.8 weight-%, preferably ≤3.0 weight-%, ≤2.5 weight-%, and most preferably ≤2.0 weight-%.

Within the given ranges of concentration for the multivalent cation salt in the fermentation broth to be subsequently centrifuged not only an improved separation result between the preciptate and the supernatant can be achieved, but simultaneously an easy removal of the multivalent cation salt can be achieved in subsequent work-up. As indicator for the scale of the removal task in respect to the removal of the multivalent cation salt a measurement of the electrical conductivity can be carried out. Like that, it was surprisingly found that within the preferred ranges of salt concentration improved separation of the solid and supernatant liquid phase could be achieved whilst at the same time the concentration could be held at a range that easy removal of the salt can be achieved in subsequent work-up steps.

In a preferred embodiment the concentration of the multivalent cation salt in the fermentation broth immediately prior to centrifugation is 0.2 weight-%, 0.3 weight-%, 0.4 weight-%, 0.5 weight-%, 0.6 weight-%, 0.7 weight-%, 0.8 weight-%, 0.9 weight-%, 1.0 weight-%, 1.1 weight-%, 1.2 weight-%, 1.3 weight-%, 1.4 weight-%, 1.5 weight-%, 1.6 weight-%, 1.7 weight-%, 1.8 weight-%, 1.9 weight-%, or 2.0 weight-%.

In one embodiment the present disclosure provides for a process as described above wherein the multivalent cation salt is CaCl₂).

Calciumchloride is widely known that is can be used in microbial fermentation processes, mostly in minor concentrations as a salt chemical in the fermentation medium. Like that, the use of calciumcholoride is already approved for food grade fermentation based production. Apart from that, the use of calciumchloride can have the advantage that not only the calcium cation but also the chloride anion can be quite easily removed from the separated liquid phase after centrifugation, for instance by ion exchange methods. There are many ion exchange resins known that offer optimized removal of calcium cations and chloride anions in the downstream work-up process. Another advantage can be seen in the non-toxic behaviour of calciumchloride towards the microbial cells. In fermentation processes where the desired HMO is separated from the biomass without lysis or other destructive measures such non-toxicity of the centrifugation aiding multivalent cation salt is beneficial in order to possibly reuse the microbial material.

In another embodiment the present disclosure provides for a process as described above wherein the multivalent cation salt is added as aqueous solution to the fermentation broth of step a) in a volume amount (v/v) ranging from ≥0.25 or ≥0.5 to ≤2.0 or ≤1.75 preferably in a volume ratio of around 1.0.

Like that, it is advantageously possible to provide an input stream to the preferably continuous centrifugation step that is well controllable and has a good flow behavior.

In another embodiment the present disclosure provides for a process as described above wherein the fermentation broth of step a) further contains at least one monosaccharide, said at least one monosaccharide being preferably selected from the group consisting of L-fucose, glucose, galactose, N-acetylglucosamine and N-acetylneuraminic acid.

In another embodiment the present disclosure provides for a process as described above wherein the fermentation broth of step a) further contains at least one further component, said at least one further component being preferably selected from the group consisting of lactose, sucrose and glycerol.

In another embodiment the present disclosure provides for a process as described above wherein the centrifugation of step c) is performed in the range of from 2,000 g to 15,000 g and/or for at least 5 min, 10 min, 15 min, 20 min, or 30 min, at a temperature of from 2 °C to 60 °C.

The minimum duration of the centrifugation treatment is dependent upon various circumstances, like applied g force, continuous or batch treatment, input volume/rate and output volumes/rates of liquid and solid phase, temperature, type of centrifuge used etc. A skilled person in the field of fermentation processes for food grade HMOs can easily find the optimum centrifugation duration for the individual gear and parameters used. Apart from that aspect, a continuous centrifugation is possible for a HMO production in industrial scale, where the biomass is emptied from the centrifuge/from the rotor after predetermined intervals. The optimum intervals for such continuous centrifugation also depend from individual circumstances of the specific gear and parameters and can be determined and applied by a skilled person.

The nature of the centrifugation parameters like high shear or low shear etc. to be chosen is dependent for instance upon the question if the desired HMO is included in the supernatant liquid phase of the fermentation broth or if the desired HMO is included in the (still intact) cells of the fermentation broth. In the first case a higher shear and higher range of g-force can be chosen, in the latter case preferably a lower shear or a lower range of g-force will be chosen in order to keep the cells intact. In a subsequent step the cells containing the desired HMO can be lysed or otherwise worked-up to release the HMO from the separated biomass. However, the core improvement of the present invention of better separation by addition of a multivalent cation salt to the fermentation broth prior to the centrifugation is achieved in both cases.

In another embodiment the present disclosure provides for a process as described above wherein the process further comprises additional steps of purifying the at least one HMO selected from one or more of the steps of
i) adding a solution of the multivalent cation salt to the centrifugation pellet obtained by step b) and centrifuging the resultant mixture to obtain a further supernatant solution;
ii) subjecting the supernatant solution of the first and optional further centrifugation steps to at least one filtration;
iii) treating the supernatant solution of the first and optional further centrifugation steps and/or the filtrate of step ii) at least one time with a cation exchange resin and/or at least one time with an anion exchange resin;
iv) subjecting the supernatant solution of the first and optional further centrifugation steps and/or the filtrate of steps ii), or iii) to at least one electrodialysis;
v) treating the supernatant solution of the first and optional further centrifugation steps and/or the filtrate of steps ii), iii), or iv) at least one time with activated charcoal; and/or
vi) subjecting a liquid process stream obtained during the recovery of the human milk oligosaccharide from the fermentation broth at least one time to a crystallization step and/or precipitation step and/or spray-drying step.

Additional purification steps for obtaining pure HMO from the centrifugation separation can be carried out in various sequences and/or repetitions suitable for the desired HMO.

Following the centrifugation step, the isolated stream is subjected to one or more purification step(s) which can include ultrafiltration, nanofiltration, anion exchange, cation exchange and decolorization, to form a purified, isolated stream. The purified, isolated stream is then treated in a concentration step and a dewatering step. These steps can be performed in any order. In other words, the purified, isolated stream can first be treated in a concentration step, and the product of the concentration then routed to a dewatering step. Alternately, the purified, isolated stream can first be subjected to a dewatering step, and the product of that dewatering step can then be routed to a concentration step. The concentration step can include evaporation, reverse-osmosis filtration and/or nanofiltration. Evaporation processes can include, e.g., falling film evaporation, climbing film evaporation and rotary evaporation, to form a concentrated, purified, isolated stream. The dewatering step can include at least one of crystallization, drying, evaporation and conventional filtration, to form an HMO product.

In step i) a repetition of the multivalent cation salt aided separation by centrifugation is carried out in order to optimize the overall yield of desired HMO by washing the centrifugation pellet (slurry) with an aqueous solution of the multivalent cation salt.

In step ii) at least one filtration step is carried out which can be preferably chosen from microfiltration, nanofiltration and/or diafiltration and/or reverse osmosis filtration.

These filtration methods are well known to the skilled person and can be chosen in relation to the purification task. For example, microfiltration and nanofiltration as work-up of process streams from an HMO producing fermentation are described in European patent application EP 3 741 77. For the purpose of this specification, the term filtration also refers to processes using plate and frame filtration, recessed chamber filtration, belt filtration, vacuum filtration, horizontal metal leaf filtration, vertical metal-leaf filtration, stacked-disc filtration, rotary vacuum filtration and combinations thereof.

In step iii) a treatment of the supernatant solution of the first and optional further centrifugation steps and/or of the filtrate of step ii) with a cation exchange resin and/or at least one time with an anion exchange resin for at least one time is carried out.

The ion exchange treatment is advantageously optimized for the removal of the multivalent cation salt and other cations and anions present in the fermentation medium. Specific cation and anion exchange resins are well known to the skilled person and such steps are, for example, described in WO 2019063757 A1.

Cation exchange treatment can be used to separate charged molecules from the HMO-containing stream. The cation exchange (CEX) stationary phase usually contains aliphatic sulfonic acid groups that are negatively charged in aqueous solution, which tightly bind any strongly basic analytes. This step removes positively charged components, e.g., residual ammonia, metals and peptides. The binding capacity of the resins used are generally 1.2 to 2.2 mmeq/L. Typical resins used for cation exchange include Dow Dowex 88, Resindion JC series such as JC603, and Resindion PK grades such as PK216.

An anion exchange step can be used to separate charged molecules. In an anion exchange column, the packing is positively charged, and therefore retains negatively charged molecules by coulombic interaction. This step removes negatively charged components, e.g., chlorides, sulphates, phosphates, organic acids, and negatively charged particles. The binding capacity of the resins used are generally 0.8 to 2.0 mmeq/L. Typical resins for anion exchange include Resindion Relite series such as RAM2, DI 82, and JA100, and Diaion WA series such as WA20.

In step iv) the supernatant solution of the first and optional further centrifugation steps and/or the filtrate of steps ii), or iii) is subjected to at least one electrodialysis.

Electrodialysis is a process for the selective removal of ions from solutions. Combined with a fermentation it can be used to separate ionic fermentation products such as organic acid and/or for desalination. Electrodialysis combines dialysis and electrolysis and can be used for the separation or concentration of ions in solutions based on their selective electromigration through semipermeable membranes.

The basic principle of electrodialysis consists of an electrolytic cell comprising a pair of electrodes submerged into an electrolyte for the conduction of ions, connected to a direct current generator. The electrode connected to the positive pole of the direct current generator is the anode, and the electrode connected to the negative pole is the cathode. The electrolyte solution then supports the current flow, which results from the movement of negative and positive ions towards the anode and cathode, respectively. The membranes used for electrodialysis are essentially sheets of porous ion-exchange material with negative or positive charge groups, and are therefore described as cationic or anionic membranes, respectively. The ion-exchange membranes are usually made of polystyrene carrying a suitable functional group (such as sulfonic acid for cationic membranes or a quaternary ammonium group for anionic membranes) cross-linked with divinylbenzene. The electrolyte can be, for example, sodium chloride, sodium acetate, sodium propionate or sulfamic acid. The electrodialysis stack is then assembled in such a way that the anionic and cationic membranes are parallel as in a filter press between two electrode blocks, such that the stream undergoing ion depletion is well separated from the stream undergoing ion enrichment (the two solutions are also referred to as the diluate (undergoing ion depletion) and concentrate (undergoing ion enrichment). The heart of the electrodialysis process is the membrane stack, which consists of several anion-exchange membranes and cation-exchange membranes separated by spacers, installed between two electrodes. By applying a direct electric current, anions and cations will migrate across the membranes towards the electrodes.

A decolorization step v) can be performed to remove color-containing components. This step can be conducted using activated carbon, such as Norit CAI activated carbon, a Hydrophobic Interaction Chromatography (HIC), or another adsorptive resin which can be functionalized, such as Resindion Relite RAD/F. The decolorization can be performed either before or after the other named purification steps.

In step vi) the process stream is at least one time subjected to a crystallization step and/or precipitation step and/or spray-drying step.

A crystallization process can be used to dewater the purified HMO material by first forming HMO nuclei in a supersaturated solution and then growing the crystals. Such a process can be either batch or continuous. Typical conditions used for the crystallization are shown in WO 2018/164937, the disclosure of which is incorporated herein by reference. The process involves concentrating a starting HMO-containing solution to a supersaturated state, and then precipitating the HMO crystal from the supersaturated solution while preferably subjecting the supersaturated solution to a temperature of at least 55 °C. Preferably, the solution has a HMO content of at least 60 wt. % and less than about 98 wt. %. Crystallization is typically limited by viscosity as the percentage of the HMO crystals grows, preferably, with a typical endpoint of around 30 wt. % crystals.

Alternatively, crystallization or precipitation of the at least one HMO from the process stream may be performed by adding a suitable amount of an organic solvent that is miscible with water to the process stream containing the at least one HMO. The organic solvent may be selected from the group consisting of C1- to C6-alcohols and C1- to C4-carboxylic acids. The step of crystallization or precipitation of the at least one HMO is not performed at the end of the recovery process such that residual amounts of the organic solvent or carboxylic acid can be removed by subsequent process steps.

A spray-drying process suitable for HMO recovery in solid form is for example described in EP 3 524 067 A1, EP 3 494 804 A1, EP 3 494 805 A1, EP 3 494 806 A1, or EP 3 494 807 A1.

In another embodiment the present disclosure provides for a process as described, wherein the aqueous solution containing the at least one HMO, and optionally the at least one monosaccharide, is spray-dried at a nozzle temperature of at least 110 °C, preferably at least 120 °C, more preferably at least 125 °C, and less than 150 °C, preferably less than 140 °C and more preferably less than 135 °C.

In another embodiment the present disclosure provides for a process as described above the fermentation broth contains the at least one HMO in an amount of less than 30% (w/v), less than 25% (w/v), or less than 20% (w/v). In an additional or alternative embodiment, the solution of any said step i) to v) or the process stream of step vi) contains the at least one HMO in an amount totaling a saccharide amount of at least 20% (w/v), 30% (w/v), or 35% (w/v), and up to 45% (w/v), 50% (w/v), or 60% (w/v).

The present invention is further directed to the use of a multivalent cation salt in the fermentative production of a human milk oligosaccharide, wherein the multivalent cation salt is added to a fermentation broth containing a human milk oligosaccharide and biomass before said fermentation broth is subjected to centrifugation for removal of the biomass.

The addition of the multivalent cation salt resulted in a better separation of biomass and other solid materials from the liquid supernatant phase and an observable lesser colorization of the liquid phase.

Preferably, the amount of the multivalent cation salt added prior to the centrifugation step is determined by optimizing the higher separation and decolorization effect versus keeping the conductivity of the liquid phase low in order to ease the subsequent removal of the cations and anions in later purification steps.

In one embodiment the present disclosure provides for a use of a multivalent cation salt in a process as described above wherein the multivalent cation salt is calcium chloride.

The present invention will be described with respect to particular embodiments, but the invention is not limited thereto but only by the claims. Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, as used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a process comprising steps A and B” should not be limited to processes consisting only of steps A and B. It means that with respect to the present invention the only relevant steps of the process are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification do not necessarily refer always to the same embodiment. Furthermore, the particular features, structures or characteristics may be combined in any suitable manner, as would be apparent to one of ordinary skill in the art from this disclosure, in one or more embodiments.

Similarly, it should be appreciated that in the description of representative embodiments of the invention, various features of the invention are sometimes grouped together in a single embodiment, figure, or description thereof for the purpose of streamlining the disclosure and facilitating the understanding of one or more of the various inventive aspects. This method of disclosure is not to be interpreted as reflecting an intention that the claimed invention requires more features than are expressly listed in each claim. Rather, as the following claims reflect, inventive aspects may require fewer than all the features of any foregoing disclosed embodiment. Thus, the claims following the detailed description are hereby expressly incorporated into this detailed description, with each claim standing on its own as a separate embodiment of this invention.

Furthermore, whereas some embodiments described herein include some but not other features included in other embodiments, combinations of features of different embodiments are meant to be within the scope of the invention, and form different embodiments, as would be understood by those familiar with the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order to facilitate the understanding of the description and drawings.

The invention will now be described by a detailed description of several embodiments of the invention. The invention being limited only by the terms of the appended claims.

### EXAMPLES

### Example 1: Purification of 2′-FL by Addition of CaCl₂

2′-FL fermentation broth was taken at the end of fermentation directly from the fermenter and used for this experiment. Five centrifuge tubes, each with 5 mL fermentation broth (fb), were each diluted with 5 ml of water (comparative sample), 0.50% CaCl₂ in water (w/w), 1.00% CaCl₂ in water (w/w) and 1.50% CaCl₂ in water (w/w). The mixtures were mixed via vortexer and centrifuged at 1800 g for 10 min at °C. The obtained solutions were decanted into a new centrifuge tube and the OD₆₀₀ and the conductivity were determined. By adding CaCl₂, a 2′-FL solution with a lower OD₆₀₀ could be obtained. This clearly indicates a better separation of the solids contained in the fermentation broth from the liquid supernatant.

TABLE 2
Conductivity and OD₆₀₀ of supernatants of 2′-FL
containing fermentation broth supplemented with CaCl₂.

| | Conductivity | |
|---|---|---|
| Sample | [μSi] | OD₆₀₀ |
| 5 mL fb + 5 mL water | 2.97 | 0.91 |
| 5 mL fb + 5 mL 0.5% | 6.23 | 0.43 |
| CaCl₂ in water(w/w) | | |
| 5 mL fb + 5 mL 1% | 9.52 | 0.32 |
| CaCl₂ in water(w/w) | | |
| 5 mL fb + 5 mL 1.5% | 12.80 | 0.34 |
| CaCl₂ in water(w/w) | | |

### Example 2: Purification of 6′-SL by Addition of CaCl₂

6′-SL fermentation broth was taken at the end of fermentation directly from the fermenter and used for this experiment. Five centrifuge tubes, each with 5.5 mL fermentation broth (fb), were each diluted with 5 ml of water (comparative sample), 5 mL of 0.50% CaCl₂ in water (w/w), 5 mL of 1.00% CaCl₂ in water (w/w) and 5 mL of 1.50% CaCl₂ in water (w/w). The mixtures were mixed via vortexer and centrifuged at 1800 g for 10 min at 10 °C. The obtained solutions were decanted into a new centrifuge tube and the OD₆₀₀ and the conductivity were determined. By adding CaCl₂, a 6′-SL solution with a lower OD₆₀₀ could be obtained. This clearly indicates a better separation of the solids contained in the fermentation broth from the liquid supernatant.

TABLE 3
Conductivity and OD₆₀₀ of supernatants of 6′-SL
containing fermentation broth supplemented with CaCl₂.

| | Conductivity | |
|---|---|---|
| Sample | [μSi] | OD₆₀₀ |
| 5 mL fb + 5 mL water | 7.8 | 17.94 |
| 5 mL fb + 5 mL 0.5% | 11.49 | 0.42 |
| CaCl₂ in water(w/w) | | |
| 5 mL fb + 5 mL 1% | 14.77 | 0.43 |
| CaCl₂ in water(w/w) | | |
| 5 mL fb + 5 mL 1.5% | 18.04 | 0.56 |
| CaCl₂ in water(w/w) | | |

### Example 3: Purification of 2′-FL by Addition of FeCl₃

2′-FL fermentation broth was taken at the end of fermentation directly from the fermenter and used for this experiment. Five centrifuge tubes, each with 5 ml fermentation broth (fb), were each diluted with 5 ml of water (comparative sample), 5 mL of 0.73% FeCl₃ in water (w/w), 5 mL of 1.46% FeCl₃ in water (w/w) and 5 mL of 2.19% FeCl₃ in water (w/w). Iron (III) chloride was used equimolar to calcium chloride in the previous experiments. The mixtures were mixed via vortexer and centrifuged at 2700 g for 10 min at room temperature. The obtained solutions were decanted into a new centrifuge tube and the OD₆₀₀ and the conductivity were determined. By adding FeCl₃, a 2′-FL solution with a lower OD₆₀₀ could be obtained. This clearly indicates a better separation of the solids contained in the fermentation broth from the liquid supernatant.

TABLE 4
Conductivity and OD₆₀₀ of supernatants of 2′-FL
containing fermentation broth supplemented with FeCl.

| | Conductivity | |
|---|---|---|
| Sample | [μSi] | OD₆₀₀ |
| 5 mL fb + 5 mL water | 3.12 | 2.05 |
| 5 mL fb + 5 mL 0.73 | 7.39 | 0.58 |
| FeCl₃ in water(w/w) | | |
| 5 mL fb + 5 mL 1.46% | 12.08 | 1.58 |
| FeCl₃ in water(w/w) | | |
| 5 mL fb + 5 mL 2.19% | 17.55 | 1.63 |
| FeCl₃ in water(w/w) | | |

1. A process for the production of a human milk oligosaccharide (HMO) derived by a microbial fermentation method, the process comprising the steps of:
a) providing a fermentation broth comprising at least one HMO and biomass;
b) adding a multivalent cation salt to the fermentation broth; and
c) subjecting the mixture of step b) to centrifugation.

2. The process according to claim 1, wherein the HMO is selected from the group consisting of 2′-FL, 3-FL, LNT, LNnT, LNFPI, LNFPII, LNFPIII, LNFPV, 3′-SL, 6′-SL, LST-a, LST-b, LST-c and DSLNT.

3. The process according to claim 1, wherein said multivalent cation salt is a divalent or trivalent cation salt.

4. The process according to claim 1, wherein the multivalent cation salt is selected from the group consisting of MgCl₂, ZnCl₂, and CaCl₂.

5. The process according to claim 1, wherein the multivalent cation salt is added to the fermentation broth of step a) in form of an aqueous solution.

6. The process according to claim 1, wherein the concentration of the multivalent cation salt in the fermentation broth after step b) is ≥0.05 weight-%.

7. The process according to claim 1, wherein the multivalent cation salt is CaCl₂.

8. The process according to claim 1, wherein the multivalent cation salt is added as aqueous solution to the fermentation broth of step a) in a volume amount (v/v) ranging from ≥0.25 or ≥0.5 to ≤2.0 or ≤1.75.

9. The process according to claim 1, wherein the fermentation broth of step a) further contains at least one monosaccharide, said at least one monosaccharide being preferably selected from the group consisting of L-fucose, glucose, galactose, N-acetylglucosamine and N-acetylneuraminic acid.

10. The process according to claim 1, wherein the fermentation broth of step a) further contains at least one further component, said at least one further component being preferably selected from the group consisting of lactose, sucrose and glycerol.

11. The process according to claim 1, wherein the centrifugation of step c) is performed in the range of from 2,000 g to 15,000 g and/or for at least 5 min, at a temperature of from 2 °C to 60 °C.

12. The process according to claim 1, wherein the process further comprises additional steps of purifying the at least one HMO selected from one or more of the steps of
i) adding a solution of the multivalent cation salt to the centrifugation pellet obtained by step b) and centrifuging the resultant mixture to obtain a further supernatant solution;
ii) subjecting the supernatant solution of the first and optional further centrifugation steps to at least one filtration;
iii) treating the supernatant solution of the first and optional further centrifugation steps and/or the filtrate of step ii) at least one time with a cation exchange resin and/or at least one time with an anion exchange resin;
iv) subjecting the supernatant solution of the first and optional further centrifugation steps and/or the filtrate of steps ii), or iii) to at least one electrodialysis;
v) treating the supernatant solution of the first and optional further centrifugation steps and/or the filtrate of steps ii), iii), or iv) at least one time with activated charcoal; and/or
vi) subjecting a liquid process stream obtained during the recovery of the human milk oligosaccharide from the fermentation broth at least one time to a crystallization step and/or precipitation step and/or spray-drying step.

13. The process according to claim 1, wherein the fermentation broth contains the at least one HMO in an amount of less than 30% (w/v), and/or the solution of any said step i) to v) or the process stream of step vi) contains the at least one HMO in an amount totaling a saccharide amount of at least 20% (w/v).

14. The process according to claim 10, wherein the aqueous solution containing the at least one HMO, and optionally the at least one monosaccharide, is spray-dried at a nozzle temperature of at least 110 °C.

15-16. (canceled)
